(19) United States
(12) Patent Application Publication
(10) Pub. No.: US 2010/0262070 A1
(43) Pub. Date: Oct. 14, 2010

(54) ULTRASONIC MEDICINE PASTE

(75) Inventor: Qing Wang, Chongqing (CN)

Correspondence Address: WOLF GREENFIELD & SACKS, P.C., 600 ATLANTIC AVENUE, BOSTON, MA, 02210-2206, US

(73) Assignee: Chongqing Ronghai Enineering Research Center of Ultrasonic Medicine Co., Ltd., Yubei District, Chongqing (CN)

(21) Appl. No.: 12/740,391

(22) PCT Filed: Sep. 1, 2008
(86) PCT No.: PCT/CN08/72219

§ 371 Date: Apr. 29, 2010

(30) Foreign Application Priority Data
Nov. 13, 2007 (CN) 200710188165.6

(51) Int. Cl. A61M 35/00; B32B 38/00

(52) U.S. Cl. 604/22; 156/278

(57) ABSTRACT

An ultrasonic medicine paste for transdermally permeating a medicine to a body and the manufacture method thereof. The medicine paste includes an adhesive layer (1) as the base layer, a medicine layer (2) adjacent to the adhesive layer (1), and ultrasonic transducer (3) for generating ultrasonic signal, a driving unit (4) that supplies an electric signal and drives the ultrasonic transducer (3) to generate ultrasonic signal, and a coating layer (5) that covers the exterior surface thereof.

The present application claims the priority to China Patent Application No. 200710188165.6 entitled “DISPOSABLE ULTRASONIC MEDICINE PASTE” filed in China on Nov. 13, 2007, the entire content of which is hereby incorporated by reference.

### FIELD OF THE INVENTION

The present invention relates to an ultrasonic medical device, and in particular, to a device for the transdermic delivery of medicaments into organisms by utilizing ultrasonic waves.

### BACKGROUND OF THE INVENTION

At present, there are three major administration routes for existing medicine therapies, i.e. injection, oral administration, and transdermal administration. Injections need to be performed intravenously or intramuscularly, and suffers from infections that may be caused. Circulated in blood, medicaments act on the suffering site in a relatively short period, and a major part of them is eliminated, rendering a substantial waste. Orally administrated medicines have a reduced efficacy after digestion and absorption by the digestive system. Also, most of medicaments are bitter in taste, which poses difficulties for the medicaments to be ingested by patients. Therefore, it has become a promising area of medicine to apply medicaments transdermally into diseased sites. Conventional topical application applies medicines directly onto affected sites by dressing or bandaging. Due to poor permeability of skins and muscle tissues, the therapeutic period is prolonged, which causes much inconvenience to patients. With the increasing development of medical science, medical devices for delivering of medicines into the body of a subject via electronic and ultrasonic means are developed, and they effectively enhance the absorption of medicaments and the therapeutic effect. Compared with electronic devices, ultrasonic devices have been more frequently utilized for being non-invasive and non-radiative. In the prior art, ultrasonic devices for transdermally delivering a medicine mainly utilizing ultrasonic atomization, heat-introduction, oscillation-introduction and the like. China Patent Application No. 90103647.1 discloses an ultrasonic device for permeating medicine solutions, in which a fabric or cotton paper soaked with the medicine solution is applied to affected sites, then an ultrasonic probe is placed thereon and moved back and forth, thereby permeating the medicine solution into tissues via High-frequency vibration, to achieve the therapeutic effect. China Patent Application No. 03802476.4 discloses an ultrasonic transdermal device, an ultrasonic transdermal auxiliary product and a method for ultrasonic transdermal delivery. The application discloses a device and method for safely and effectively permeating medicines to the sites to be treated through skin, muscle and fat. Furthermore, ultrasonic waves with different frequency are used for permeating medicines into different sites. However, the prior art disclosed techniques only employ ultrasonic wave as a means for improving medicine permeation, and ultrasonic waves and medicines are not considered as an entity, not to mention the integration or miniaturization thereof.

To sum up, the ultrasonic transdermal delivery devices in the prior art suffer from disadvantages such as the complexity in structure, being inconvenient to be used by patients on their own, medicines being discreted with the ultrasonic system, which tends to cause off-target and in turn damage to surrounding tissues, and using the same probe to treat several patients, which tends to cause cross-contamination, or the like. The integrative ultrasonic medicine paste according to the present invention completely solves the above problems. Besides, it presents a low-cost and is suitable to be produced and commercialized in a large scale.

### SUMMARY OF THE INVENTION

The present invention overcomes the above disadvantages and provides an ultrasonic medicine paste. The medicine paste is simple in structure, and is easy to use and prepare.

The technical solution used to solve the technical problems according to the present invention is as follows:

the ultrasonic medicine paste comprises an adhesive layer as the base layer, a medicine layer immediately adjacent to the adhesive layer, an ultrasonic transducer generating ultrasonic signals, a driving unit providing electric signals and driving the ultrasonic transducer to generate ultrasonic signals, and a coating layer that covers the exterior surfaces and forms the paste into one entity.

The adhesive layer is made of flexible materials with good permeability, such as perforated cloth, either of the surfaces of which is coated with an adhesive for avoiding cross contamination. The surface opposite to the one coated with the adhesive has two surface regions, that is, the first region and the second region.

The surface of the adhesive layer, on which an adhesive is coated, is further attached with a release film. During treatment, the release film can be peeled off, and the paste can be attached to the sites to be treated.

The medicine layer, made of an adsorbing material sprayed with or soaked in the medicines, is fixed on the first region through pressing and adhering and the like. Immediately adjacent to the medicine layer is the ultrasonic wave emitting surface of the ultrasonic transducer.

The driving unit is located on the back of the ultrasonic transducer, and comprises an ultrasonic driving circuit and a power supply, integrated or not.

When the power supply is integrated in the paste, it is disposed in the same layer as the ultrasonic driving circuit, and on the back of the ultrasonic transducer. The power supply is electrically connected with the ultrasonic transducer and the ultrasonic driving circuit, being switched off by an on/off film, one end of which being exposed to the outside of the paste. A part of the coating layer integratively covers the medicine layer, the ultrasonic transducer and the driving unit, and fixes them on the first region of the adhesive layer, while the other part is combined with the second region of the adhesive layer, to form a freely flexible bonding edge. In operation, the on/off film is withdrawn, and the power supply is switched on to operate the paste.

When the power supply is placed outside the paste, it comprises an external battery box, a battery, a power lead, a plug and the fixed socket disposed on the paste. The battery is placed in the battery box, and connected to the plug via the power lead. The fixed socket on the paste is electrically connected with the ultrasonic driving circuit and the ultrasonic transducer. A part of the coating layer integratively covers the medicine layer, the ultrasonic transducer and the driving unit, and fixes them on the first region of the adhesive layer, while the other part is combined with the second region of the adhesive layer to form a freely flexible bonding edge. In operation, the plug is inserted in the fixed socket, and the power supply is switched on to operate the paste.

Corresponding to the present ultrasonic paste, a method for manufacturing the paste is also provided, comprising:

coating an adhesive on an adhesive layer, the surface opposite to the surface coated with the adhesive having two surface regions, the first region and the second region, the adhesive layer being the lowest layer of the present paste, and being made of a flexible material having good permeability;

fixing a medicine layer, an ultrasonic transducer, a driving unit on the first region;

coating the second region with an adhesive and combining it with the coating layer;

a strongly adsorbing material is sprayed with or soaked in a medicine, to form the medicine layer;

Fixing the medicine layer on the first region of the adhesive layer, with the ultrasonic wave emitting surface immediately attached to the ultrasonic transducer;

placing an on/off film to keep the power supply off, one end of the on/off film being exposed to the outside of the paste, to facilitate being withdrawn in operation, to switch on the power supply;

integratively covering the medicine layer, the ultrasonic transducer and the driving unit and fixing them on the first region of the adhesive layer with a part of the coating layer, while the other part of the coating layer is combined with the second region of the adhesive layer to form a freely flexible bonding edge, so that the paste can fit on an unflat skin surface of a human body.

The present invention has the following advantages: the present invention skillfully incorporates the ultrasonic system into the medicine paste, greatly improving the utility of the paste, while keeping the paste light, convenient and disposable, so that the pates can be put into clinical use sooner and better. The present invention is simple in structure, easy to manufacture, and convenient to use, and can avoid adverse outcomes caused by injections, such as infection, pain, and fear. The present structure enables the ultrasonic paste to be used in the same way as conventional pastes, thus addresses the key problem that hinders the ultrasonic medicine permeating technologies from being wiled used in clinic.

### BRIEF DESCRIPTION OF THE DRAWINGS

FIG. 1 is the schematic view of the present invention.

FIG. 2 is the sectional view of example 1.

FIG. 3 is the sectional view of example 2.

FIG. 4 is the schematic view of external power supply of example 2.

FIG. 5 is the schematic view of a contour of the bonding edge of the present paste.

FIG. 6 is the schematic view of another contour of the bonding edge of the present paste.

Reference signs: 1. adhesive layer; 2. medicine layer; 3. ultrasonic transducer; 4. driving unit; 41. power supply; 42. ultrasonic driving circuit; 5. coating layer; 7. on/off film; 11. the first region; 12. the second region; 60. battery box; 61. socket; 62. plug; 63. power lead; 64. battery.

### DETAILED DESCRIPTION OF EMBODIMENTS

The structures of the present invention and the effects can be achieved will be further described below in detail in connection with the drawings.

### Example 1

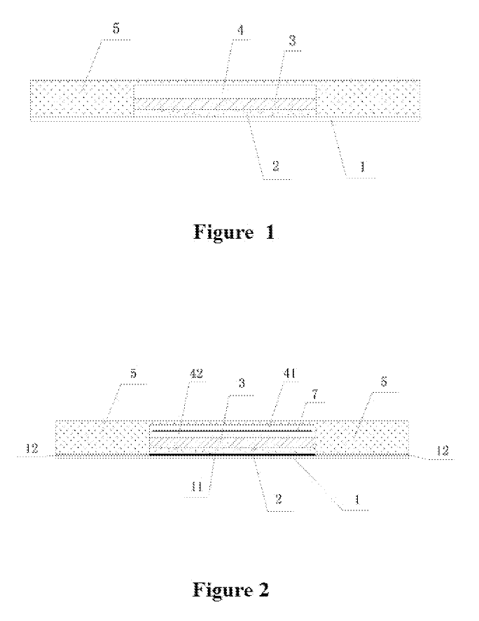

As shown in FIG. 1, the ultrasonic medicine paste according to the present invention comprises an adhesive layer 1, a medicine layer 2, a ultrasonic transducer 3, a driving unit 4, and a coating layer 5. The medicine layer 2, ultrasonic transducer 3 and driving unit 4 are sequentially laminated from bottom to top, with coating layer 5 covering the three layers. Adhesive layer 1 is the base layer.

As shown in FIG. 2, adhesive layer 1 is the lowest layer of the present paste, and is made of a flexible material having good permeability. In this example, the adhesive layer 1 is made of perforated cloth, either of the surfaces of which is coated with an adhesive for one-time attachment. The other surface of the adhesive layer has two regions, a first region 11 which is preferably located at the center of the cloth and used to fix medicine layer 2, ultrasonic transducer 3 and driving unit 4 thereon; and a second region 12 which is preferably located on the peripheral of the cloth, and can be coated with an adhesive for adhering to the coating layer. The paste can also be made in a manner that the first region 11 is not located at the center, and the second region 12 is asymmetrical, facilitating the treatment of specific sites.

The medicine layer 2 is made of a strongly adsorbing material, which has been sprayed with a medicine thereon, or has been soaked with a medicines. In this example, nonwoven fabrics that has been soaked with a medicine solution are preferred. The medicine layer is fixed on the first region 11 of the adhesive layer 1. Immediately attached to the medicine layer is the ultrasonic wave emitting surface of the ultrasonic transducer 3.

In the example, the driving unit 4 comprises a power supply 41 and an ultrasonic driving circuit 42, which is located on the back surface of the ultrasonic transducer 3. The power supply 41 is electronically connected respectively to the ultrasonic driving circuit 42 and the ultrasonic transducer 3, which is preferably a battery. An on/off film 7 is used to keep the power supply in opening state, and one of its ends is exposed to the outside of the paste, facilitating the withdrawing thereof in operation, so as to switch on the power supply.

A part of the coating layer 5 integrally covers the medicine layer 2, the ultrasonic transducer 3 and the driving unit 4, and fixes them on the first region 11 of the adhesive layer 1, while the other part is combined with the second region 12 of the adhesive layer 1 to form a freely flexible bonding edge, so that the paste can fit an unflat skin surface of a patient.

Furthermore, this example comprises a disposable release film, which is attached on the surface of the adhesive layer being coated with the adhesive, and can be disposed upon use.

When using the medicine paste of the present invention for treatment, the release film is firstly peeled off, and the paste is bonded via the adhesive layer 1 onto the skin surface of the sites requiring treatment. Subsequently, the on/off film 7 is withdrawn by pulling its end exposed to the outside, to switch on the power supply, so that the ultrasonic transducer 3, driven by the ultrasonic driving circuit 42, emits ultrasonic wave toward the medicine layer 2. Thus the solution of the medicine is permeated through the adhesive layer 1 having a good permeability, the skin of the body, the tissues and the like and into the affected sites, where the medicine acts and achieve the treatment.

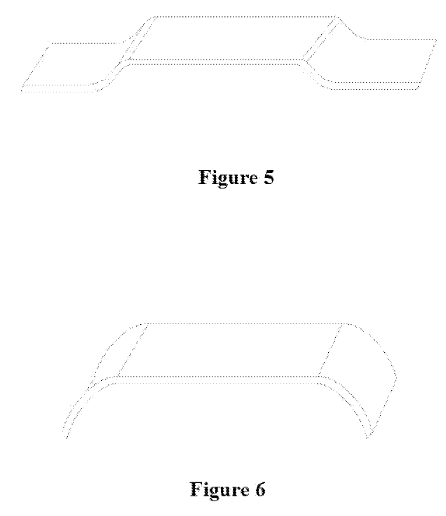

FIGS. 5 and 6 are schematic views of bended bonding edges formed by combining the coating layer 5 and the second region 12 on the adhesive layer 1. However, the contour of the bonding edges is not limited thereto. Since the present medicine paste is small in size and simple, it can be easily attached on affected skins during treatment, and can be peeled off after treatment.

### Example 2

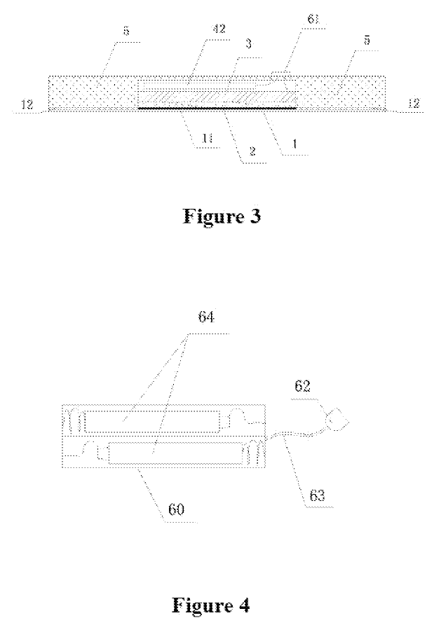

As shown in FIGS. 3 and 4, in order to further minimize the medicine paste, this example differs from example 1 in that the power supply 41 in the driving unit 4 is not integrated. The external power supply 41 comprises a battery box 60, a battery 64, a power lead 63, a plug 62, and fixed socket 61 disposed on the paste. The battery 64 is placed in the battery box 60 and is connected to the plug 62 via the power lead 63 to form an electric pathway. The fixed socket 61 is electrically connected to the driving unit 42 and the ultrasonic transducer 3. In this example, a part of the coating layer 5 covers the medicine layer 2, the ultrasonic transducer 3 and the ultrasonic driving unit 42 and fixes them on the first region 11 of the adhesive layer 1, while the other part is combined with the second region 12 of the adhesive layer 1 to form a freely flexible bonding edge. When using the paste, the plug 63 is inserted into the socket 64 to switch on the power supply, and the paste begins to work.

It will be appreciated by those skilled in the art that the ultrasonic transducer can be selected from those having different working frequencies, and the number of the ultrasonic transducers is not limited to only one. In stead, an appropriate number of ultrasonic transducers can be selected as needed, so that the paste can work under different frequencies. The ultrasonic transducers having different frequencies can operate alternately or simultaneously.

The means to switch on/off of the power supply of the ultrasonic transducer is not limited to the on/off film. Other known means in the art can also be used without departing from the scope of the present invention.

Corresponding to the two examples described above, the present invention also provides a method for manufacturing an ultrasonic paste, comprising the steps of:

300. coating an adhesive onto one surface of an adhesive layer, the other surface having two surface regions, the first region 11 and the second region 12. The adhesive layer 1 is the lowest layer of the present paste, and is made of a flexible material having good permeability. In this example, adhesive layer 1 is made of perforated cloth, with either of its surfaces being coated with an adhesive for one-time attachment;

301. fixing the medicine layer 2, ultrasonic transducer 3 and driving unit 4 on the first region 11, the first region 11 preferably being located at the center of the paste;

302. coating the second region with an adhesive and combining it with the coating layer; the second region 12 preferably being a peripheral region, and can be coated with an adhesive for combining with the coating layer. The paste can also be made so that the first region 11 is not located at the center, and the second region 12 is asymmetrical, facilitating the treatment on sites with specific shapes;

303. making a medicine layer 2 by applying to a strongly adsorbing material a medicine, through spraying or soaking. Nonwoven fabrics soaked with a medicine solution are preferred;

304. fixing the medicine layer onto the first region 11 of the adhesive layer 1. Immediately attached to the medicine layer is the ultrasonic wave emitting surface of the ultrasonic transducer 3.

In the example, driving unit 4 comprises a power supply 41 and an ultrasonic driving circuit 42, which is located on the back surface of the ultrasonic transducer 3. The power supply 41 is electronically connected to the ultrasonic driving circuit 42 and the ultrasonic transducer 3, and is preferably a battery. An on/off film 7 is used to keep the power supply in opening state, and one of its ends is exposed to the outside of the paste, facilitating the withdrawing thereof in operation, so as to switch on the power supply;

305. integrally covering the medicine layer 2, the ultrasonic transducer 3 and the driving unit 4 with a part of the coating layer 5, and fixes them on the first region 11 of the adhesive layer 1, while the other part of the coating layer 5 is combined with the second region 12 of the adhesive layer 1 to form a freely flexible bonding edge, so that the paste can fit an unflat skin surface of a patient.

Furthermore, this example comprises a release film, which is attached on the surface of the adhesive layer that is coated with adhesive, and can be disposed upon use.

When using the medicine paste of the present invention for treatment, the release film is firstly peeled off, and the paste is bonded via the adhesive layer 1 onto the skin surface of the sites requiring treatment. Subsequently, the on/off film 7 is withdrawn by pulling its end exposed to the outside, to switch on the power supply, so that the ultrasonic transducer 3, driven by the ultrasonic driving circuit 42, emits ultrasonic wave toward the medicine layer 2. Thus the solution of the medicine is permeated through the adhesive layer 1 having a good permeability, the skin of the body, the tissues and the like and into the affected sites, where the medicine acts and achieve the treatment.

As shown in FIGS. 3 and 4, in order to further minimize the medicine paste, the power supply 41 in the driving unit 4 is not integrated. The external power supply 41 comprises a battery box 60, a battery 64, a power lead 63, a plug 62, and fixed socket 61 disposed on the paste. The battery 64 is placed in the battery box 60 and is connected to the plug 62 via the power lead 63 to form an electric pathway. The fixed socket 61 is electrically connected to the driving unit 42 and the ultrasonic transducer 3. In this example, a part of the coating layer 5 covers the medicine layer 2, the ultrasonic transducer 3 and the ultrasonic driving unit 42 and fixes them on the first region 11 of the adhesive layer 1, while the other part is combined with the second region 12 of the adhesive layer 1 to form a freely flexible bonding edge. When using the paste, the plug 63 is inserted into the socket 64 to switch on the power supply, and the paste begins to work.

It will be appreciated by those skilled in the art that, various medicines can be soaked into the paste and permeated into organisms, depending on the therapeutic needs and the specific conditions (such as pharyngitis, osteoarticular diseases, painful muscle, skin diseases, acupuncture treatments in traditional Chinese medicine, and the like) or health care needs (such as cosmetology, whitening, wrinkle removing and weight controlling). The ultrasonic medicine pastes disclosed in the examples of the present invention are characterized by miniaturization and integration, in which the size of the ultrasonic transducer and the driving unit is comparable to that of a round button cell, and the thickness thereof is only about 3-4 times that of the cell. The volume thereof is only about 1/(10˜1000) of the ultrasonic medicine permeating device in the prior art, while the intensity and duration of the ultrasonic waves emitted can sufficiently meet the needs for medicine permeation. When the technology according to the present invention is applied to deliver analgesics to some of the patients suffering from painful muscle and bone fracture, an obvious analgesic effect can be achieved within only 10-20 minutes, 3-5 times faster than simply applying the medicines.

The ultrasonic paste provided by the present invention has been described in detail, and the principle and embodiments of the present invention have been illustrated by specific examples. However, the illustration of the above examples is made merely for better understanding the present method and its core concept. Modifications in specific embodiments and applications can be made by those skilled in the art based on the spirit of the present invention. To sum up, the disclosure of the present invention should not be considered as any limitation to the present invention.

1. An ultrasonic medicine paste comprising: an adhesive layer as the base layer, a medicine layer immediately adjacent to the adhesive layer, an ultrasonic transducer generating an ultrasonic signal, a driving unit providing an electric signal and driving the ultrasonic transducer to generate the ultrasonic signal, and a coating layer that covers the exterior surface of the paste.

2. The ultrasonic medicine paste according to claim 1, wherein the adhesive layer is made of a flexible material having a good permeability.

3. The ultrasonic medicine paste according to claim 1 or 2, wherein the adhesive layer is made of perforated cloth.

4. The ultrasonic medicine paste according to claim 1 or 2, wherein a release film is attached to the surface of the adhesive layer, on which an adhesive is coated.

5. The ultrasonic medicine paste according to claim 1, wherein the medicine layer is made of an adsorbing material, onto which a medicine is sprayed, or which has been soaked with a medicine.

6. The ultrasonic medicine paste according to claim 2, wherein one surface of the flexible material is coated with an adhesive; and the opposite surface comprise two surface regions, the first region and the second region.

7. The ultrasonic medicine paste according to claim 1, wherein the coating layer is made of a flexible material, a part of which is used for covering the medicine layer, the ultrasonic transducer and the driving unit, while the other part is combined with the second region of the adhesive layer to form a freely flexible bonding edge.

8. The ultrasonic medicine paste according to claim 1, wherein the driving unit comprises a power supply and an ultrasonic driving circuit, the power supply being placed inside the paste or separately outside the paste.

9. The ultrasonic medicine paste according to claim 8, wherein the power supply is arranged inside the paste, integrated with the ultrasonic driving circuit, and disposed on the back of the ultrasonic transducer; the power supply is electrically connected to the ultrasonic transducer and the ultrasonic driving circuit, being switched off by an on/off film, wherein one end of the film is exposed to the outside of the paste for being withdrawn in operation, so as to switch on the power supply to operate the paste.

10. The ultrasonic medicine paste according to claim 8, wherein the power supply is arranged outside the paste, and comprises an external battery box, a battery, a power lead, a plug and the a fixed socket disposed on the paste, the battery being placed in the battery box and connected to the plug via the power lead; the fixed socket on the paste being electrically connected to the ultrasonic driving circuit and the ultrasonic transducer; wherein the plug is inserted in the fixed socket in operation, so as to switch on the power supply to operate the paste.

11. A method for manufacturing an ultrasonic medicine paste, comprising the steps of:
coating one surface of an adhesive layer with an adhesive, wherein the opposite surface of the adhesive layer comprises two surface regions, the first region and the second region, the adhesive layer being the lowest layer of the paste, and being made of a flexible material having good permeability;
fixing a medicine layer, an ultrasonic transducer and a driving unit on the first region;
coating the second region with an adhesive and combining it with the coating layer;
spraying a medicine onto an adsorbing material, or soaking an adsorbing material with a medicine, to form a medicine layer;
fixing the medicine layer on the first region, with the ultrasonic wave emitting surface of the ultrasonic transducer immediately attached to the medicine layer;
using an on/off film to keep the power supply off, wherein one end of the on/off film is exposed to the outside of the paste for being withdrawn in operation, to switch on the power supply;
integrally covering the medicine layer, the ultrasonic transducer and the driving unit and fixing them on the first region of the adhesive layer with a part of the coating layer, and combining the other part with the second region of the adhesive layer to form a freely flexible bonding edge, so that the paste can fit an unflat skin surface of a human body.

12. The method according to claim 11, wherein the first region is not located at the center of the adhesive layer, and the second region is an asymmetrical peripheral.
